(19) United States
(12) Patent Application Publication
(10) Pub. No.: US 2024/0390194 A1
(43) Pub. Date: Nov. 28, 2024

(54) FLUID COLLECTION ASSEMBLIES INCLUDING ONE OR MORE LEAK PREVENTION FEATURES

(71) Applicant: PUREWICK CORPORATION, Covington, GA (US)

(72) Inventor: Jill W. Jones, Avondale Estates, GA (US)

(21) Appl. No.: 18/694,090

(22) PCT Filed: Sep. 21, 2022
(86) PCT No.: PCT/US2022/044243

§ 371 Date: Mar. 21, 2024

Related U.S. Application Data
(60) Provisional application No. 63/247,478, filed on Sep. 23, 2021.

(51) Int. Cl. A61F 13/475; A61F 13/45; A61F 13/64; A61M 1/00; A61M 39/10

(52) U.S. Cl. CPC A61F 13/475; A61F 13/64; A61M 1/71; A61M 39/105; A61F 2013/4506; A61M 2205/0266; A61M 2210/1092; A61M 2210/1475

(57) ABSTRACT

An example fluid collection assembly includes a fluid impermeable layer including a proximal end region, a distal end region opposite the proximal end region, a front region defining an opening, and a back region opposite the front region. The fluid impermeable layer also defines at least a chamber and a fluid outlet. The fluid collection assembly also includes at least one porous material disposed in the chamber that extends across the opening. The fluid collection assemblies includes one or more leak prevention features that are configured to decrease the likelihood that bodily fluids leak from the fluid collection assembly. The leak prevention features may include one or more of at least a portion the porous material extending across the opening exhibiting a wedge-like shape, the fluid outlet being positioned on the back region, or an extension extending from the distal end region.

### CROSS-REFERENCE TO RELATED APPLICATION(S)

This application claims priority to U.S. Provisional Patent Application No. 63/247,478 filed on Sep. 23, 2021, the disclosure of which is incorporated herein, in its entirety, by this reference.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

In an embodiment, a fluid collection assembly is disclosed. The fluid collection assembly includes a fluid impermeable layer including a proximal end region, a distal end region spaced from the proximal end region, a front region defining an opening, and a back region opposite the front region. The fluid impermeable layer defines at least a chamber and a fluid outlet. The fluid collection assembly also includes at least one porous material disposed in the chamber and extending across the opening. The fluid collection assembly includes one or more leak prevention features. The one or more leak prevention features includes at least one of at least a portion of the at least one porous material extending across the opening exhibiting a generally wedge-like shape, the fluid outlet positioned on the back region of the fluid impermeable layer, an extension extending from the distal end region of the fluid impermeable layer. The extension is configured to be positioned in a gluteal cleft.

In an embodiment, a fluid collection system is disclosed. The fluid collection system includes a fluid collection assembly. The fluid collection assembly includes a fluid impermeable layer including a proximal end region, a distal end region opposite the proximal end region, a front region defining an opening, and a back region opposite the front region. The fluid impermeable layer defines at least a chamber and a fluid outlet. The fluid collection assembly also includes at least one porous material disposed in the chamber and extending across the opening. The fluid collection assembly includes one or more leak prevention features. The one or more leak prevention features includes at least one of at least a portion of the at least one porous material extending across the opening exhibiting a generally wedge-like shape, the fluid outlet positioned on the back region of the fluid impermeable layer, an extension extending from the distal end region of the fluid impermeable layer. The extension is configured to be positioned in a gluteal cleft. The fluid collection system also includes a fluid storage container and a vacuum source. The chamber of the fluid collection assembly, the fluid storage container, and the vacuum source are in fluid communication with each that, when one or more bodily fluids are present in the chamber, a suction provided from the vacuum source to the chamber of the fluid collection assembly removes the one or more bodily fluids from the chamber and deposits the bodily fluids in the fluid storage container.

In an embodiment, a fluid collection system is disclosed. The fluid collection system includes a fluid collection assembly. The fluid collection assembly includes a fluid impermeable layer including a proximal end region, a distal end region opposite the proximal end region, a front region defining an opening, and a back region opposite the front region. The fluid impermeable layer defines at least a chamber and a fluid outlet. The fluid collection assembly also includes at least one porous material disposed in the chamber and extending across the opening. The fluid collection assembly includes one or more leak prevention features. The one or more leak prevention features includes at least one of at least a portion of the at least one porous material extending across the opening exhibiting a generally wedge-like shape, the fluid outlet positioned on the back region of the fluid impermeable layer, an extension extending from the distal end region of the fluid impermeable layer. The extension is configured to be positioned in a gluteal cleft. The fluid collection system also includes generally wedge shaped pillow configured to fit between legs. The generally wedge shaped pillow includes an apex configured to be positioned proximate to a urethral opening and to press the portion of the fluid permeable outer layer extending across the opening against the urethral opening.

In an embodiment, a fluid collection system is disclosed. The fluid collection system includes The fluid collection assembly includes a fluid impermeable layer including a proximal end region, a distal end region opposite the proximal end region, a front region defining an opening, and a back region opposite the front region. The fluid impermeable layer defines at least a chamber and a fluid outlet. The fluid collection assembly also includes at least one porous material disposed in the chamber and extending across the opening. The fluid collection assembly includes one or more leak prevention features. The one or more leak prevention features includes at least one of at least a portion of the at least one porous material extending across the opening exhibiting a generally wedge-like shape, the fluid outlet positioned on the back region of the fluid impermeable layer, an extension extending from the distal end region of the fluid impermeable layer. The extension is configured to be positioned in a gluteal cleft. The fluid collection system also includes a leg attachment device configured to be attached to a leg and a portion of at least one conduit that is disposed outside of the chamber.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C, respectively, according to an embodiment.

FIG. 2 is a cross-sectional schematic of a fluid collection assembly, according to an embodiment.

FIG. 3 is a cross-sectional schematic of a fluid collection assembly, according to an embodiment.

FIG. 4A is an isometric view of a fluid collection assembly, according to an embodiment.

FIG. 4B is a cross-sectional schematic of the fluid collection assembly taken along plane 4B-4B as illustrated in FIG. 4A, according to an embodiment.

FIG. 5 is a cross-sectional schematic of a fluid collection assembly, according to an embodiment.

FIG. 6A is a top view of a fluid collection system that includes a fluid collection assembly and a pillow being used with an individual, according to an embodiment.

FIG. 6B is a cross-sectional schematic of the pillow, according to an embodiment.

FIG. 7 is a top view of a fluid collection system that includes a fluid collection assembly and a leg attachment device being used with an individual, according to an embodiment.

FIG. 8 is a cross-sectional schematic of a conduit, according to an embodiment.

FIG. 9 is a top plan view of a fluid collection assembly that includes an extension, according to an embodiment.

FIG. 10 is a block diagram of a fluid collection system for fluid collection, according to an embodiment.

### DETAILED DESCRIPTION

Embodiments are directed to fluid collection assemblies, fluid collection assemblies including the same, and methods of using the same. An example fluid collection assembly includes a fluid impermeable layer (e.g., fluid impermeable barrier) including a proximal end region, a distal end region spaced from the proximal end region, a front region defining at least one opening, and a back region opposite the front region. The fluid impermeable layer also defines at least a chamber and a fluid outlet. The fluid collection assembly also includes at least one porous material disposed in the chamber. A portion of the porous material extends across the opening. The fluid collection assemblies includes one or more leak prevention features that are configured to decrease the likelihood that one or more bodily fluids (e.g., urine, blood, sweat, etc.) leak from the fluid collection assembly. The leak prevention features may include one or more of at least a portion of the porous material extending across the opening exhibiting a wedge-like shape, the fluid outlet positioned on the back region of the fluid impermeable layer, or an extension extending from the distal end region that is configured to be positioned in the gluteal cleft.

The fluid collection assemblies disclosed herein (e.g., fluid collection assemblies including the one or more leak prevention features) are examples of female fluid collection assemblies that are configured to collect bodily fluids (e.g., urine, blood, sweat, etc.) from a vaginal region of an individual. During use, the fluid collection assemblies disclosed herein may be positioned on a vaginal region of an individual such that at least a portion of the porous material extending across the opening is positioned adjacent to the individual's urethral opening. The individual may discharge urine the bodily fluids from the urethral opening. The bodily fluids may be received through the opening, into the porous material, and into the chamber. The bodily fluids may be removed from the chamber via at least one conduit that is in fluid communication with the chamber.

The fluid collection assemblies disclosed herein exhibit one or more improvements over conventional fluid collection assemblies (e.g., fluid collection assemblies that do not include the one or more leak prevention features disclosed herein). The ability of conventional fluid collection assemblies to prevent leaks may depend on the correct placement of the conventional fluid collection assemblies on the vaginal region of the individual and securing the conventional fluid collection assemblies to the vaginal region of the individual. However, variations in the anatomy between different individuals makes placement and securement of the conventional fluid collection assemblies to the vaginal region difficult. In an example, conventional fluid collection assemblies may rely on contact between the thighs of the individual and the conventional fluid collection assemblies to secure the conventional fluid collection assemblies to the vaginal region. However, individuals with relatively small thighs (e.g., a thin individual) may make insufficient contact to adequately secure the conventional fluid collection assemblies to the vaginal region which may cause leakage. In an example, conventional fluid collection assemblies may have a conduit extending from a proximal end region thereof. In such an example, the conduit does not serve any purpose in securing the conventional fluid collection assemblies to the vaginal region and may apply a torque to the rest of the conventional fluid collection assemblies if the conduit is bent. The torque applied to the rest of the conventional fluid collection assemblies may cause such assemblies to move relative to the vaginal region thereby causing leakage. In an example, a distal end region of the conventional fluid collection assemblies may be disposed in the gluteal cleft to align the conventional fluid collection assemblies relative to the vaginal region and help secure the conventional fluid collection assemblies to the vaginal region. However, positioning the distal end region of the conventional fluid collection assemblies in the gluteal cleft may increase the likelihood that stool migrates from the anal regions of the individual to the opening of the conventional fluid collection assemblies which, in turn, may increase the likelihood of a urinary tract infection. Further, positioning the distal end region of the conventional fluid collection assemblies in the gluteal cleft may increase the likelihood that an inlet of the conduit becomes at least partially obstructed thereby decreasing the rate at which bodily fluids may be removed from the conventional fluid collection assemblies. The decreased rate at which the bodily fluids are removed from the conventional fluid collection assemblies may cause a backup of the bodily fluids in the conventional fluid collection assemblies which, in turn, may cause the bodily fluids to leak.

The fluid collection assemblies disclosed herein include one or more leak prevention features that resolve at least some of these issues associated with conventional fluid collection assemblies. In an example, the one or more leak prevention features may include at least a portion of the porous material extending across the opening exhibiting a wedge-like shape. The wedge-like shape of the porous material allows a portion of the porous material to be disposed between the labia folds of the individual. Positioning the porous material between the labia folds allows the labia folds to align relative to and secure the fluid collection assembly to the individual, even when the individual has relatively small thighs. In an example, the one or more leak prevention features may include the fluid outlet positioned on the back region of the fluid impermeable layer. In such an example, the ability of the conduit to apply a torque that can move the fluid collection assembly relative to the vaginal region is diminished compared to a substantially similar fluid collection assembly that had the fluid outlet positioned on the proximal end region. In an example, the one or more leak prevention features may include an extension extending from the distal end region that is configured to be positioned in the gluteal cleft to help align and secure the fluid collection assembly to the vaginal region.

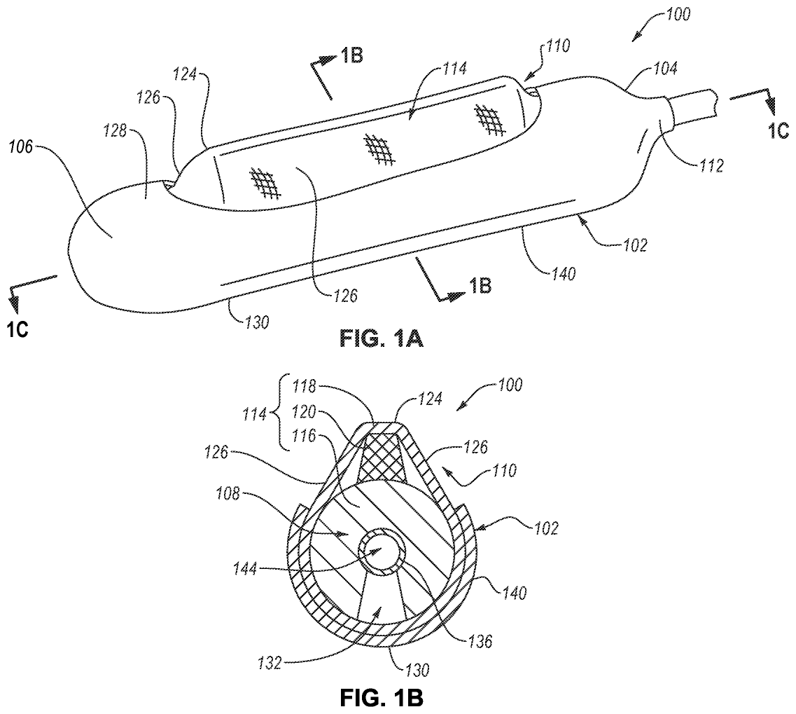

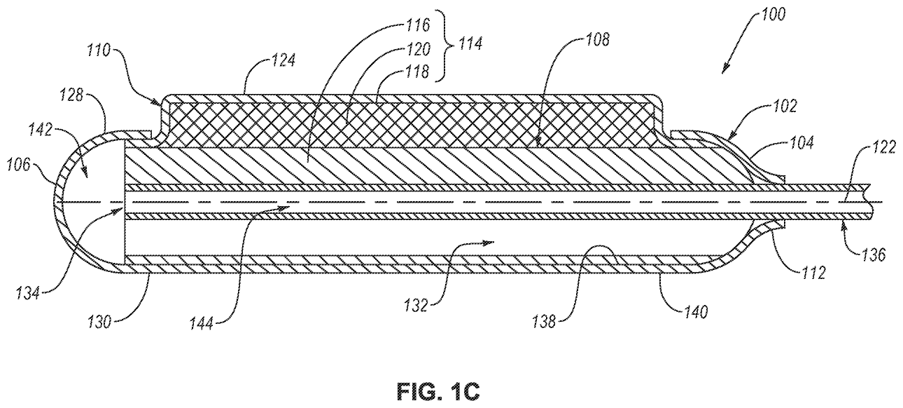

FIG. 1A is an isometric view of a fluid collection assembly 100, according to an embodiment. FIGS. 1B and 1C are cross-sectional schematics of the fluid collection assembly 100 taken along planes 1B-1B and 1C-1C, respectively, according to an embodiment. The fluid collection assembly 100 includes a fluid impermeable layer 102. The fluid impermeable layer 102 includes a proximal end region 104 and a distal end region 106 spaced from the proximal end region 104. The fluid impermeable layer 102 also defines at least a chamber 108, at least one opening 110, and a fluid outlet 112. The fluid collection assembly 100 also includes at least one porous material 114 disposed in the chamber 108. In the illustrated embodiment, the porous material 114 includes a fluid permeable inner layer 116 (e.g., fluid permeable support), a fluid permeable outer layer 118 (e.g., fluid permeable membrane), and at least one fluid permeable intermediate material 120 though it is noted that the porous material 114 may include fewer or more elements.

In the illustrated embodiment, the one or more leak prevention features of the fluid collection assembly 100 include at least a portion of the porous material 114 that extends across the opening 110 exhibiting a protruding configuration, such as wedge-like shape. For simplicity and brevity, the portion of the porous material that extends across the opening 110 and exhibits the wedge-like shape will be referred to as the wedge portion. The wedged portion may extend outwardly from the rest of the fluid collection assembly 100 (e.g., extend further from a central, longitudinal axis 122 of the fluid collection assembly 100 than the portions of the fluid impermeable layer 102 that define the opening 110). The wedged portion improves the connection between the porous material and the individual using the fluid collection assembly 100. For example, the wedged portion may be more easily fit between the labia folds of the individual than if the portions of the porous material 114 that extends across the opening 110 exhibited a generally semi-cylindrical shape. The better fit of the wedged portion allows more of the porous material 114 to be disposed between the labia folds and increases the comfortability of the fluid collection assembly 100 than if the portions of the porous material 114 that extends across the opening 110 exhibited a generally semi-cylindrical shape. The better fit allows the wedged portion to better align the fluid collection assembly 100 with the vaginal region of the individual and better secure the fluid collection assembly 100 to the vaginal region than if the porous material 114 did not include the wedge portion.

The wedged portion may exhibit an apex 124 and two sides 126 tapering from the apex 124. In other words, the distance between the two sides 126 may increase with increasing distance from the apex 124. The apex 124 and the two sides 126 may cause the portion of the porous material 114 that includes the wedged portion to exhibit a variety of cross-sectional shapes. In an embodiment, when the portions of the porous material 114 that do not include the wedged portion exhibit a generally circular cross-sectional shape (e.g., exhibit a generally cylindrical shape), the portion of the porous material 114 that includes the wedged portion may exhibit a generally tear-drop cross-sectional shape when the two sides 126 are generally straight. In an embodiment, when the portions of the porous material 114 that do not include the wedged portion exhibit a generally circular cross-sectional shape, the portion of the porous material 114 that includes the wedged portion may exhibit a generally almond-like cross-sectional shape when the two sides 126 are generally convexly curved. In an embodiment, the wedged portion may exhibit a generally semi-oval or ellipsoidal cross-sectional shape.

The percentage of the wedged portion that is positioned between the labia folds may depend on the size of the labia folds due to the tapered shaped thereof. For example, a smaller percentage of the wedged portion may be disposed between relatively small labia folds (which may be difficult or impossible with a semi-spherical porous material) while a greater percentage (e.g., all) of the wedged portion may be disposed between relatively large labia folds. Thus, the wedged portion may be used effectively with different individuals that each exhibit differently sized labia folds.

The tapered shape of the wedged portion may allow the wedged portion to be positioned closer to the urethral opening of the individual than if the porous material 114 did not include the wedged portion. For example, the tapered shape of the wedged portion may allow the apex 124 of the wedged portion to be positioned adjacent to or, more preferably, abut the urethral opening of the individual. Positioning the porous material 114 closer to the urethral opening of the individual increases the percentage of the bodily fluids discharged from the urethral opening that are received into the porous material 114 which, in turn, decreases the quantity of bodily fluids that leak from the porous material 114.

In an embodiment, as previously discussed, the porous material 114 includes the fluid permeable inner layer 116 (e.g., a fluid permeable support), the fluid permeable outer layer 118 (e.g., a fluid permeable membrane), and the fluid permeable intermediate material 120 that is distinct from the fluid permeable inner layer 116 and the fluid permeable outer layer 118. In such an embodiment, the fluid permeable intermediate material 120 is positioned between the portions of the fluid permeable inner layer 116 and the fluid permeable outer layer 118 that are adjacent to the opening 110. Such a position of the fluid permeable intermediate material 120 causes the porous material 114 to exhibit the generally wedge-like shape.

The fluid permeable intermediate material 120 may be formed from any suitable fluid permeable material otherwise the fluid permeable intermediate material 120 may form an obstacle to any bodily fluids flowing through the opening 110. In an example, the fluid permeable intermediate material 120 may be formed from an elastic fluid permeable material. As used herein, an elastic fluid permeable material includes any material that may return to its shape after being compressed. Forming the fluid permeable intermediate material 120 from the elastic fluid permeable material may allow the fluid permeable intermediate material 120 to continuously apply a normal force to the labia folds that helps secure the porous material 114 to the labia folds. It also allows the fluid permeable intermediate material 120 to be pitched before positioning the wedged portion between the labia folds which may facilitate positioning the wedged portion between the labia folds. In an example, the fluid permeable intermediate material 120 may be formed from a gauze, a soft fabric, a woven or nonwoven material, a porous polymer structure, an open cell foam, spun nylon fiber, paper, or any other suitable fluid permeable material.

In an embodiment, the fluid permeable intermediate material 120 is formed from the fluid permeable inner layer 116. In such an embodiment, a cutout is formed in a portion of the fluid permeable inner layer 116 that is spaced from the opening 110. For example, the fluid impermeable layer 102 may include a front region 128 that defines the opening 110 and a back region 130 that is opposite the front region 128. The cutout may be formed from a portion of the fluid permeable inner layer 116 that is adjacent to or at least proximate to the back region 130. The cutout may then be positioned between the portions of the fluid permeable inner layer 116 and the fluid permeable outer layer 118 that are adjacent to the opening 110. In other words, the cutout forms the fluid permeable intermediate material 120. Forming the fluid permeable intermediate material 120 from the fluid permeable inner layer 116 may decrease the quantity of material that is used to form the porous material 114 since no additional material is necessary to form the fluid permeable intermediate material 120.

Forming the fluid permeable intermediate material 120 from the fluid permeable inner layer 116 may form a drainage channel 132 in the chamber 108 where the cutout would have been located. The drainage channel 132 is defined by the fluid permeable inner layer 116 and is generally unoccupied space. As previously discussed, the cutout may be formed in a portion of the fluid permeable inner layer 116 that is adjacent to or at least proximate to the back region 130 of the fluid impermeable layer 102. Generally, during use, none or a small percentage of the bodily fluids that enter the chamber 108 flows in the portions of the porous material 114 adjacent to the back region 130 such that forming the drainage channel 132 has little to no effect on the flow of the bodily fluids in the chamber 108. However, when the fluid collection assembly 100 receives a large volume of bodily fluids in a short period of time, a non-negligible volume of bodily fluids may flow into the drainage channel 132. The fact that the drainage channel 132 is unoccupied allows the drainage channel 132 to receive a larger quantity of bodily fluids than if the drainage channel 132 was occupied by a porous material thereby allowing the chamber 108 to receive a larger volume of bodily fluids. Since the drainage channel 132 is spaced from the opening 110, the fact that the drainage channel 132 is unoccupied does not increase the likelihood that the bodily fluids leak from the chamber 108. Further, the bodily fluids in the drainage channel 132 may flow towards the inlet 134 of the conduit 136 faster than if the drainage channel 132 was occupied by a porous material assuming the inlet 134 is gravimetrically downstream from the drainage channel 132 thereby allowing quicker removal of the bodily fluids from the chamber 108. When the inlet 134 is not gravimetrically downstream from the drainage channel 132, the bodily fluids may remain in the drainage channel 132 until the bodily fluids in adjacent portions of the porous material 114 are removed, at which point the bodily fluids in the drainage channel 132 may be received in the porous material 114. The bodily fluids received in the porous material 114 may then flow towards the inlet 134. It is noted that the fluid permeable intermediate material 120 may be integrally formed with the fluid permeable inner layer 116, as shown in FIG. 3.

As previously discussed, the fluid collection assembly 100 includes the porous material 114 disposed in the chamber 108. The porous material 114 may cover at least a portion (e.g., all) of the opening 110. The porous material 114 is exposed to the environment outside of the chamber 108 through the opening 110. In an embodiment, the porous material 114 may be configured to wick any bodily fluids away from the opening 110, thereby preventing the bodily fluids from escaping the chamber 108. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the porous material 114. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the porous material 114 (e.g., absorbency), such as less than about 30 wt % of the dry weight of the porous material 114, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the porous material 114. The porous material 114 may also wick the bodily fluids generally towards an interior of the chamber 108, as discussed in more detail below. In an embodiment, the porous material 114 may include at least one absorbent or adsorbent material.

In an embodiment, at least a portion of the porous material 114 (e.g., one or more of the fluid permeable outer layer 118, the fluid permeable intermediate material 120, or, more preferably, the fluid permeable inner layer 116) may be hydrophobic. The porous material 114 may be hydrophobic when the porous material 114 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the porous material 114 may limit absorption, adsorption, and solubility of the bodily fluids in the porous material 114 thereby decreasing the amount of bodily fluids held in the porous material 114. In an embodiment, at least a portion of the porous material 114 is hydrophilic. In an embodiment, the fluid permeable inner layer 116 and/or the fluid permeable intermediate material 120 is more hydrophobic (e.g., exhibits a larger contact angle with water) than the fluid permeable outer layer 118. The lower hydrophobicity of the fluid permeable outer layer 118 may help the porous material 114 receive the bodily fluids from the urethral opening while the greater hydrophobicity of the fluid permeable inner layer 116 and/or the fluid permeable intermediate material 120 limits the bodily fluids that are retained in the porous material 114.

In an embodiment, the porous material 114 may include the fluid permeable outer layer 118 at least partially disposed in the chamber 108. The fluid permeable outer layer 118 may cover at least a portion (e.g., all) of the opening 110. The fluid permeable outer layer 118 may be composed to wick the bodily fluids away from the opening 110, thereby preventing the bodily fluids from escaping the chamber 108.

In an embodiment, the fluid permeable outer layer 118 may include any material that may wick the bodily fluids. For example, the fluid permeable outer layer 118 may include fabric, such as a gauze (e.g., a silk, linen, or cotton gauze), another soft fabric, another smooth fabric, a nonwoven material, or any of the other porous materials disclosed herein. Forming the fluid permeable outer layer 118 from gauze, soft fabric, and/or smooth fabric may reduce chaffing caused by the fluid collection assembly 100.

The fluid collection assembly 100 may include the fluid permeable inner layer 116 at least partially disposed in the chamber 108. The fluid permeable inner layer 116 is configured to support the fluid permeable outer layer 118 since the fluid permeable outer layer 118 may be formed from a relatively foldable, flimsy, or otherwise easily deformable material. For example, the fluid permeable inner layer 116 may be positioned such that the fluid permeable outer layer 118 is disposed between the fluid permeable inner layer 116 and the fluid impermeable layer 102. As such, the fluid permeable inner layer 116 may support and maintain the position of the fluid permeable outer layer 118. The fluid permeable inner layer 116 may include any material that may wick, absorb, adsorb, or otherwise allow fluid transport of the bodily fluids, such as any of the fluid permeable outer layer materials disclosed herein above. For example, the fluid permeable outer layer material(s) may be utilized in a more dense or rigid form than in the fluid permeable outer layer 118 when used as the fluid permeable inner layer 116. The fluid permeable inner layer 116 may be formed from any fluid permeable material that is less deformable than the fluid permeable outer layer 118. For example, the fluid permeable inner layer 116 may include a porous polymer (e.g., nylon, polyester, polyurethane, polyethylene, polypropylene, etc.) structure or an open cell foam, such as spun nylon fiber. In some examples, the fluid permeable inner layer 116 may include a nonwoven material. In some examples, the fluid permeable inner layer 116 may be formed from a natural material, such as cotton, wool, silk, or combinations thereof. In such examples, the material may have a coating to prevent or limit absorption of fluid into the material, such as a water repellent coating. In some examples, the fluid permeable inner layer 116 may be formed from fabric, felt, gauze, or combinations thereof.

The fluid impermeable layer 102 at least partially defines a chamber 108 (e.g., interior region) and an opening 110. For example, the interior surface(s) 138 of the fluid impermeable layer 102 at least partially defines the chamber 108 within the fluid collection assembly 100. The fluid impermeable layer 102 temporarily stores the bodily fluids in the chamber 108. The fluid impermeable layer 102 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable layer 102 substantially prevents the bodily fluids from passing through the fluid impermeable layer 102. In an example, the fluid impermeable layer 102 may be air permeable and fluid impermeable. In such an example, the fluid impermeable layer 102 may be formed of a hydrophobic material that defines a plurality of pores. At least one or more portions of at least an outer surface 140 902 of the fluid impermeable layer 102 may be formed from a soft and/or smooth material, thereby reducing chaffing.

In some examples, the fluid impermeable layer 102 may be tubular (ignoring the opening), such as substantially cylindrical (as shown), oblong, prismatic, or flattened tubes. During use, the outer surface 140 902 of the fluid impermeable layer 102 may contact the individual. The fluid impermeable layer 102 may be sized and shaped to fit between the labia and/or the gluteal cleft between the legs of a female user.

The opening 110 provides an ingress route for bodily fluids to enter the chamber 108. The opening 110 may be defined by the fluid impermeable layer 102 such as by an inner edge of the fluid impermeable layer 102. For example, the opening 110 is formed in and extends through the fluid impermeable layer 102, from the outer surface 140 to the inner surface 138 902, thereby enabling bodily fluids to enter the chamber 108 from outside of the fluid collection assembly 100.

The opening 110 may be an elongated hole in the fluid impermeable layer 102. For example, the opening 110 may be defined as a cut-out in the fluid impermeable layer 102. The opening 110 may be located and shaped to be positioned adjacent to a female urethra. The opening 110 may have an elongated shape because the space between the legs of a female is relatively small when the legs of the female are closed, thereby only permitting the flow of the bodily fluids along a path that corresponds to the elongated shape of the opening 110 (e.g., longitudinally extending opening).

The fluid collection assembly 100 may be positioned proximate to the female urethral opening and the bodily fluids may enter the chamber 108 of the fluid collection assembly 100 via the opening 110. The fluid collection assembly 100 is configured to receive the bodily fluids into the chamber 108 via the opening 110. When in use, the opening 110 may have an elongated shape that extends from a first location below the urethral opening (e.g., at or near the anus or the vaginal opening) to a second location above the urethral opening (e.g., at or near the top of the vaginal opening or the pubic hair).

In some examples, the fluid impermeable layer 102 may define a fluid outlet 112 sized to receive the conduit 136. The at least one conduit 136 may be disposed in the chamber 108 via the fluid outlet 112. The fluid outlet 112 may be sized and shaped to form an at least substantially fluid tight seal against the conduit 136 or the at least one tube thereby substantially preventing the bodily fluids from escaping the chamber 108.

The porous material 114 may at least substantially completely fill the portions of the chamber 108 that are not occupied by the conduit 136. In some examples, the porous material 114 may not substantially completely fill the portions of the chamber 108 that are not occupied by the conduit 136. In such an example, the fluid collection assembly 100 includes the reservoir 142 disposed in the chamber 108.

The reservoir 142 is a substantially unoccupied portion of the chamber 108. The reservoir 142 may be defined between the fluid impermeable layer 102 and one or both of the fluid permeable outer layer 118 and fluid permeable inner layer 116. The bodily fluids that are in the chamber 108 may flow through the fluid permeable outer layer 118 and/or fluid permeable inner layer 116 to the reservoir 142. The reservoir 142 may retain the bodily fluids therein.

The bodily fluids that are in the chamber 108 may flow through at least one of the fluid permeable inner layer 116, the fluid permeable outer layer 118, or the fluid permeable intermediate material 120 to the reservoir 142. The fluid impermeable layer 102 may retain the bodily fluids in the reservoir 142. While depicted in the distal end region 106, the reservoir 142 may be located in any portion of the chamber 108 such as the proximal end region 104. The reservoir 142 may be located in a portion of the chamber 108 that is designed to be located in a gravimetrically low point of the fluid collection assembly when the fluid collection assembly is worn.

In some examples (not shown), the fluid collection assembly 100 may include multiple reservoirs, such as a first reservoir that is located at the portion of the chamber 108 closest to the inlet of the conduit 136 (e.g., distal end region 106) and a second reservoir that is located at the portion of the of the chamber 108 that is at or near proximal end region 104). In another example, the fluid permeable inner layer 116 is spaced from at least a portion of the conduit 136, and the reservoir 142 may be the space between the fluid permeable inner layer 116 and the conduit 136.

The conduit 136 may be at least partially disposed in the chamber 108. The conduit 136 may be used to remove the bodily fluids from the chamber 108. The conduit 136 includes at least one wall defining an inlet 134, an outlet (not shown) downstream from the inlet 134, and a passageway 144. The outlet of the conduit 136 may be operably coupled to a vacuum source, such as a vacuum pump for withdrawing fluid from the chamber 108 through the conduit 136. For example, the conduit 136 may extend into the fluid impermeable layer 102 from the proximal end region 104 and may extend to the distal end region 106 to a point proximate to the reservoir 142 therein such that the inlet 134 is in fluid communication with the reservoir 142. The conduit 136 fluidly couples the chamber 108 with the fluid storage container (not shown) or the vacuum source (not shown).

The conduit 136 may extend through a bore in the porous material 114. In an embodiment, the conduit 136 extends from the fluid outlet 112, through the bore, to a location that is proximate to the reservoir 142. In such an embodiment, the inlet 134 may not extend into the reservoir 142 and, instead, the inlet 134 may be disposed within the porous material 114 (fluid permeable outer layer 118 and/or fluid permeable inner layer 116) or at a terminal end thereof. For example, an end of the conduit 136 may be coextensive with or recessed within the fluid permeable outer layer 118 and/or fluid permeable inner layer 116. In an embodiment, the conduit 136 is at least partially disposed in the reservoir 142 and the inlet 134 may be extended into or be positioned in the reservoir 142. In an embodiment, the inlet 134 may be at of the reservoir 142. The bodily fluids collected in the fluid collection assembly 100 may be removed from the chamber 108 via the conduit 136.

Locating the inlet 134 at or near a location expected to be the gravimetrically low point of the chamber 108 when worn by an individual enables the conduit 136 to receive more of the bodily fluids than if inlet 134 was located elsewhere and reduces the likelihood of pooling (e.g., pooling of the bodily fluids may cause microbe growth and foul odors). For instance, the bodily fluids in the fluid permeable outer layer 118 and the fluid permeable inner layer 116 may flow in any direction due to capillary forces. However, the bodily fluids may exhibit a preference to flow in the direction of gravity, especially when at least a portion of the fluid permeable outer layer 118 and/or the fluid permeable inner layer 116 is saturated with the bodily fluids. Accordingly, one or more of the inlet 134 or the reservoir 142 may be located in the fluid collection assembly 100 in a position expected to be the gravimetrically low point in the fluid collection assembly 100 when worn by an individual, such as the distal end region 106.

The inlet 134 and the outlet of the conduit 136 are configured to fluidly couple (e.g., directly or indirectly) the vacuum source (not shown) to the chamber 108 (e.g., the reservoir 142). As the vacuum source (FIG. 9) applies a vacuum/suction in the conduit 136, the bodily fluids in the chamber 108 (e.g., at the distal end region 106 such as in the reservoir 142) may be drawn into the inlet 134 and out of the fluid collection assembly 100 via the conduit 136. In some examples, the conduit 136 may be frosted or opaque (e.g., black) to obscure visibility of the bodily fluids therein.

As previously discussed, the conduit 136 may be configured to be at least insertable into the chamber 108. In an example, the conduit 136 may be positioned in the chamber 108 such that a terminal end of the conduit 136 is spaced from the fluid impermeable layer 702 or other components of the fluid collection assembly 100 that may at least partially obstruct or block the inlet 134. Further, the inlet 134 of the conduit 136 may be offset relative to a terminal end of the porous material 114 such that the inlet 134 is closer to the proximal end region 104 of the fluid collection assembly 100 than the terminal end of the porous material 114. Offsetting the inlet 134 in such a manner relative to the terminal end of the porous material 114 allows the inlet 134 to receive bodily fluids directly from the porous material 114 and, due to hydrogen bonding, pulls more bodily fluids from the porous material 114 into the conduit 136.

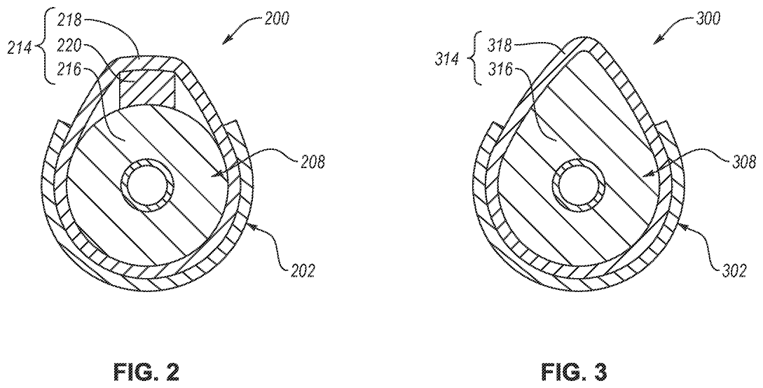

The fluid permeable intermediate materials disclosed herein, in some embodiments, may not be formed from the fluid permeable inner layer. For example, FIG. 2 is a cross-sectional schematic of a fluid collection assembly 200, according to an embodiment. Except as otherwise disclosed herein, the fluid collection assembly 200 is the same or substantially similar to any of the fluid collection assemblies disclosed herein. The fluid collection assembly 200 includes a fluid impermeable layer 202 defining a chamber 208 and at least one porous material 214 disposed in the chamber 208. In an embodiment, as illustrated, the porous material 214 includes a fluid permeable inner layer 216, a fluid permeable outer layer 218, and a fluid permeable intermediate material 220. The fluid permeable intermediate material 220 is distinct from the fluid permeable inner layer 216 and the fluid permeable outer layer 218. The fluid permeable intermediate material 220 is not formed from the fluid permeable inner layer 216. As such, optionally, the fluid permeable inner layer 216 may not include a drainage passageway and/or the fluid permeable intermediate material 220 may be formed from a material that is different than the fluid permeable inner layer 216.

In some embodiments, the fluid permeable intermediate material may be omitted from the porous materials disclosed herein. For example, FIG. 3 is a cross-sectional schematic of a fluid collection assembly 300, according to an embodiment. Except as otherwise disclosed herein, the fluid collection assembly 300 is the same or substantially similar to any of the fluid collection assemblies disclosed herein. The fluid collection assembly 300 includes a fluid impermeable layer 302 defining a chamber 308 and at least one porous material 314 disposed in the chamber 308. In an embodiment, as illustrated, the porous material 314 includes a fluid permeable inner layer 316 and a fluid permeable outer layer 318. The porous material 314 does not include a fluid permeable intermediate material. Instead, at least one of the fluid permeable inner layer 316 or the fluid permeable outer layer 318 exhibits a cross-sectional shape (e.g., a generally teardrop-like or almond-like cross-sectional shape) that forms a wedge-like shape.

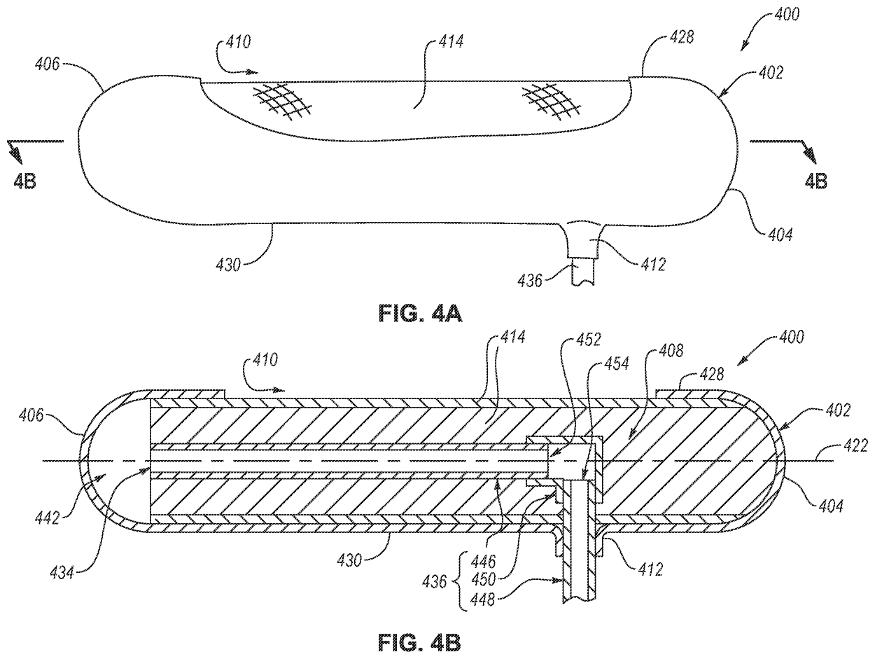

As previously discussed, the fluid collection assemblies disclosed herein may include one or more leak prevention features other than or in addition to the wedge-like shape of the porous material discussed above. For example, the fluid collection assemblies disclosed herein may include a fluid outlet on a back region of the fluid impermeable layer instead of on the proximal end region (as shown in FIG. 1A). FIG. 4A is an isometric view of a fluid collection assembly 400, according to an embodiment. FIG. 4B is a cross-sectional schematic of the fluid collection assembly 400 taken along plane 4B-4B as illustrated in FIG. 4A, according to an embodiment. Except as otherwise disclosed herein, the fluid collection assembly 400 may be the same or substantially similar to any of the fluid collection assemblies disclosed herein. For example, the fluid collection assembly 400 may include a fluid impermeable layer 402 having a proximal end region 404, a distal end region 406 opposite the proximal end region 404, a front region 428 defining an opening 410, and a back region 430 opposite the front region 428. The fluid impermeable layer 402 also defines a chamber 408 and a fluid outlet 412. The fluid collection assembly 100 also includes at least one porous material 414 disposed in the chamber 408 that extends across the opening 410. The fluid collection assembly 400 may also include at least one conduit 436 in fluid communication with the chamber 408 via the fluid outlet 412.

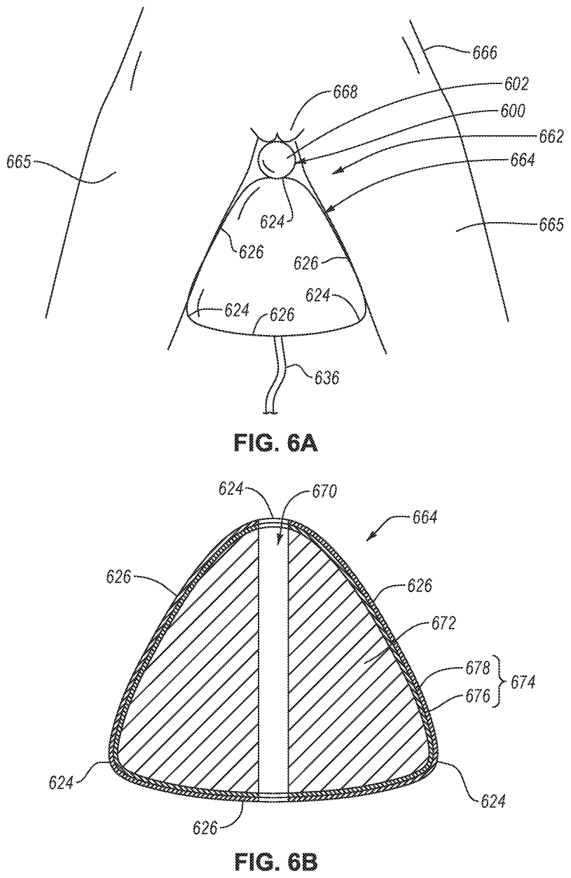

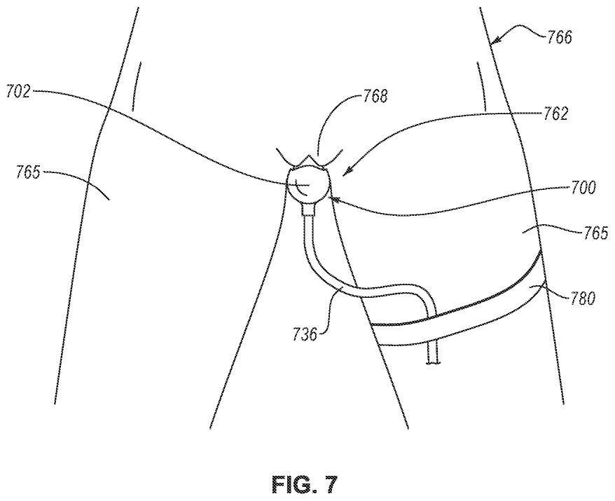

The fluid outlet 412 is positioned on or near the back region 430 of the fluid impermeable layer 402. Positioning the fluid outlet 412 on or near the back region 430 allows the conduit 436 to extend from the fluid impermeable layer 402 in a direction that, during use, is generally between or adjacent to the legs of the individual. Such positioning the conduit 436 limits any torque that is applied to the rest of the fluid collection assembly 400 when the conduit 436 bends and, when the conduit 436 does apply a torque, decreases the likelihood that the torque causes the fluid collection assembly 400 to move. Further, positioning the conduit 436 between the legs of the individual allows the conduit 436 to secure the fluid collection assembly 400 to the individual. For example, the conduit 436 may contact the legs (e.g., thighs) of the individual which provides another contact point that secures the fluid collection assembly 400 to the individual. Further, as will be discussed in more detail with regards to FIG. 7, the conduit 436 may exhibit a natural shape and the conduit 436 may be shaped to exhibit a bent shape that is different than the natural shape. Since the conduit 436 extends from the back region 430, the desire of the conduit 436 to return to the natural shape thereof may press the fluid collection assembly 400 into the vaginal region. Also, positioning the fluid outlet 412 on the back region 430 of the fluid impermeable layer 402 allows the fluid collection assembly 400 to be used with the securement devices illustrated in FIGS. 6A-7 (e.g., the pillow 664 and the leg attachment device 780).

During use, the urethral opening of the individual is positioned adjacent to a portion of the opening 410 that is closer to the proximal end region 404 of the fluid impermeable layer 402 than a distal end region 406. Such a position of the urethral opening relative to the opening 410 may require any bodily fluids that are initially not received by the porous material 414 to have to flow down most of the length of the opening 410 without being received by the porous material 414 before such bodily fluids may leak. In an embodiment, the fluid outlet 412 may be formed in a portion of the back region 430 that is closer to the proximal end region 404 of the fluid impermeable layer 402 than a distal end region 406 of the fluid impermeable layer 402. For example, the fluid outlet 412 may be positioned on a portion of the back region 430 that is directly opposite or nearly directly opposite the portion of the opening 410 that the urethral opening is positioned against. As such, any force applied from the conduit 436 to the fluid collection assembly 400 (e.g., by the conduit 436 desiring to change from a bent shape to a natural shape) presses the porous material 414 into the urethral opening and the vaginal region. Pressing the porous material 414 into the urethral opening and the vaginal region may decrease the volume of bodily fluids that are not received into the porous material 414 (e.g., that leak).

During use, at least some of the bodily fluids may generally flow towards the distal end region 406 of the fluid impermeable layer 402 since, generally, the distal end region 406 is the gravimetric low point of the chamber 408. As such, the inlet 434 of the conduit 436 may be positioned in or adjacent to the distal end region 406 (e.g., in or adjacent to the fluid reservoir 442). Positioning the inlet 434 of the conduit 436 in or adjacent to the distal end region 406 while the fluid outlet 412 is positioned on the back region 430 may require a bend to be formed in the conduit 436. In an embodiment, the conduit 436 includes a first conduit 446, a second conduit 448, and a fitting 450 (e.g., an L-shaped fitting). The first conduit 446 and the second conduit 448 are attached together using the fitting 450 and the fitting 450 may form the bend. The first conduit 446 may define the inlet 434 and a first outlet 452 opposite and downstream from the inlet 434. The first conduit 446 may extend in a direction that is generally parallel to a longitudinal axis 422 of the fluid collection assembly 400 which allows the inlet 434 to be positioned at or near the distal end region 406 (e.g., in or near the reservoir 442). The second conduit 448 may define an additional inlet 454 and a second outlet (not shown) opposite and downstream from the additional inlet 454. The second conduit 448 may extend in a direction that is generally perpendicular or at least oblique relative to the longitudinal axis 422. The fitting 450 includes a first opening (not labeled, occupied by first conduit 446), a second opening (not labeled, occupied by second conduit 448) downstream from the first opening, and a bend between the first and second opening. The first conduit 446 may be positioned in or otherwise in fluid communication with the first opening and the second conduit 448 may be positioned in or otherwise in fluid communication with the second opening. As such, the fitting 450 allows the first outlet 452 to be in fluid communication with the additional inlet 454 and allows the conduit 436 to exhibit a bend therein without kinking. It has been found that forming the bend in the conduit 436 with the first conduit 446, the second conduit 448, and the fitting 450 has little to no effect on the vacuum provided to the chamber 408 and the rate at which bodily fluids may be removed from the chamber 408. It is also noted that the porous material 414 may define a first bore that is configured to receive the first conduit 446 and a second bore configured to receive the second conduit 448.

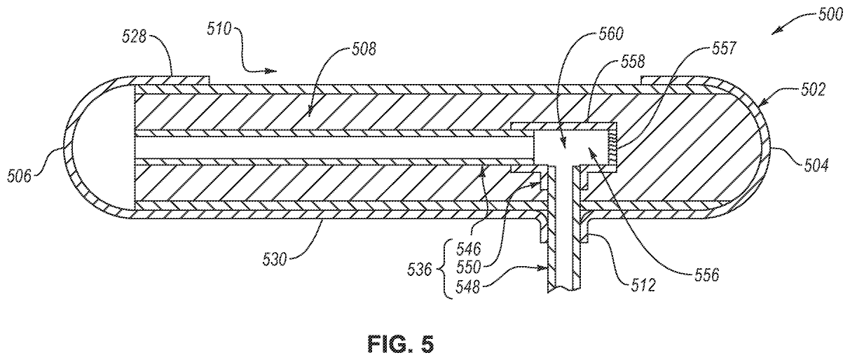

FIG. 5 is a cross-sectional schematic of a fluid collection assembly 500, according to an embodiment. Except as otherwise disclosed herein, the fluid collection assembly 500 may be the same or substantially similar to any of the fluid collection assemblies disclosed herein. For example, the fluid collection assembly 500 may include a fluid impermeable layer 502 having a proximal end region 504, a distal end region 506 opposite the proximal end region 504, a front region 528 defining an opening 510, and a back region 530 opposite the front region 528. The fluid impermeable layer 502 also defines a chamber 508 and a fluid outlet 512 positioned on the back region 530 of the fluid impermeable layer 502.

The fluid collection assembly 500 includes at least one conduit 536 in fluid communication with the chamber 508. The conduit 536 includes a bend therein. In an embodiment, the conduit 536 includes a first conduit 546, a second conduit 548, and a fitting 550 in fluid communication with the first conduit 546 and the second conduit 548. The fitting 550 allows the conduit 536 to exhibit a bend between the first and second conduits 546, 548. However, unlike the fitting 450 illustrated in FIG. 4B, the fitting 550 is a T-shaped fitting. For example, the fitting 550 may include a first opening (not labeled, occupied by the first conduit 546), a second opening 556 opposite the first opening, and a third opening (not labeled, occupied by the second conduit 548) between the first opening and the second opening 556. The first opening includes the first conduit 546 positioned therein or otherwise fluidly coupled thereto and the third opening includes the second conduit 548 positioned therein or otherwise fluidly coupled thereto. In an embodiment, as shown, the second opening 556 is closed (e.g., with a plug 557 or cap) such that bodily fluids or air cannot flow out of the second opening 556. In an embodiment, the second opening 556 may include an additional conduit disposed therein and the additional conduit is closed (e.g., with a plug or cap) such that bodily fluids and air cannot flow out of the additional conduit. It is noted that the second opening 556 is still considered closed when the additional conduit is disposed in the second opening 556 so long as the additional conduit is closed.

At least the second opening 556 of the fitting 550 is formed from one or more walls 558 extending from a common portion 560 of the fitting 550. The walls 558 extending from the common portion 560 provide support for the portions of the fluid collection assembly 500 proximate to the proximal end region 504. For example, referring to FIG. 4B, the portion of the fluid collection assembly 400 proximate to the proximal end region 404 is only occupied by the porous material 414. It has been found that the portion of the fluid collection assembly 400 proximate to the proximal end region 404 may be too flimsy to remain in contact with the vaginal region of the individual since the fluid impermeable layer 402 and the porous material 414 may not provide sufficient structure. However, referring back to FIG. 5, the walls 558 of the fitting 550 provide additional structure to the portions of the fluid collection assembly 500 proximate to the proximal end region 504 such that such portions of the fluid collection assembly 500 are not too flimsy. The additional conduit, if present, extending from the second opening 556 may also provide additional structure to the portions of the fluid collection assembly 500 proximate to the proximal end region 504.

It is noted that the conduits illustrated in FIGS. 4A-5 are merely two examples of conduits including a bend. In other examples, conduits including a bend that may be used in any of the fluid collection assemblies disclosed herein include a conduit that is merely bent (e.g., an external force is required to maintain the bend) or a conduit having a bend formed therein (e.g., no external force is required to maintain the bend).

As previously discussed, positioning the fluid outlet on the back region of the fluid impermeable layer allows the fluid collection assembly to be used with one or more securement devices. FIGS. 6A-7 illustrate different examples of securement devices that may be used with a fluid collection assembly that includes the fluid outlet on the back region of the fluid impermeable layer. FIG. 6A is a top view of a fluid collection system 662 that includes a fluid collection assembly 600 and a pillow 664 being used with an individual 666, according to an embodiment. Except as otherwise disclosed herein, the fluid collection assembly 600 may be the same or substantially similar to any of the fluid collection assemblies disclosed herein. For example, the fluid collection assembly 600 may include a fluid impermeable layer 602 defining a fluid outlet on a back region thereof.

The pillow 664 is configured to fit between the legs 665 of the individual 666. As such, at least a portion of the pillow 664 may exhibit a generally wedge-shaped (e.g., triangular) cross-sectional shape which allows the pillow 664 to be positioned comfortably between the legs 665 of the individual 666. For example, the pillow 664 may include one or more apexes 624 and sides 626 extending from the apexes 624. One of the apexes 624 may be positioned to be proximate to the vaginal region 668 of the individual 666. The apex 624 proximate to the vaginal region 668 may press the fluid collection assembly 600 into the vaginal region 668. Pressing the apex 624 into the fluid collection assembly 600 may help secure the fluid collection assembly 600 to the vaginal region 668 thereby preventing leaks. Due to the compressibility of the pillow 664, it is noted that the apex 624 that contacts the fluid collection assembly 600 does not need to define a recess, hole, or other receptacle that is configured to receive the fluid collection assembly 600, though the apex 624 may define such a receptacle.

During use, the pillow 664 may contact the legs 665 (e.g., thighs) of the individual 666. Contacting the pillow 664 to the legs 665 of the individual 666 may help secure the pillow 664 between the legs 665 of the individual 666.

FIG. 6B is a cross-sectional schematic of the pillow 664, according to an embodiment. As shown in FIG. 6B, the pillow 664 may define a recess 670 that is configured to receive at least the conduit 636 (shown in FIG. 6A) that extends from the back region of the fluid impermeable layer 602. The recess 670 may allow the conduit 636 to extend from the back region of the fluid impermeable layer 602. The recess 670 may also form an anchor that helps secure the pillow 664 to the fluid collection assembly 600.

The pillow 664 includes a compressible supporting member 672 (e.g., an enclosed envelope containing stuffing). Optionally, the pillow 664 may include a casing 674 positioned or positionable over the compressible supporting member 672. In an embodiment, the casing 674 may include a fluid impermeable layer 676. The fluid impermeable layer 676 may prevent any bodily fluids that leak from the fluid collection assembly 600 from soiling the supporting member 672. The casing 674 may be easier to clean than the supporting member 672 (e.g., machine washable or wipeable with a disinfecting wipe) or may be disposable thereby decreasing the need to clean the supporting member 672. In an embodiment, the casing 674 includes a fluid receiving layer 678 disposed on an outer surface of the fluid impermeable layer 676 (e.g., a surface of the fluid impermeable layer 676 opposite the surface that contacts the supporting member 672). The fluid receiving layer 678 may be configured to receive at least some of the bodily fluids that leak from the fluid collection assembly 600 thereby preventing or at least limiting the soiling of bedding, clothing, etc. The fluid receiving layer 678 may also receive sweat discharged from the thighs of the individual thereby keeping the individual dry.

FIG. 7 is a top view of a fluid collection system 762 that includes a fluid collection assembly 700 and a leg attachment device 780 being used with an individual 766, according to an embodiment. Except as otherwise disclosed herein, the fluid collection assembly 700 may be the same or substantially similar to any of the fluid collection assemblies disclosed herein. For example, the fluid collection assembly 700 may include a fluid impermeable layer 702 defining a fluid outlet on a back region thereof.

The leg attachment device 780 is configured to be secured to a leg 765 of the individual 766. In an example, as illustrated, the leg attachment device 780 includes at least one strap (e.g., elastic band, a rope or string, a belt, or other suitable strap) that wraps around the leg 765 to secure the leg attachment device 780 to the individual. In an example, the leg attachment device 780 includes at least one adhesive, such as tape, that secures the leg attachment device 780 to the individual 766. In an example, the leg attachment device 780 includes an elastic band. The leg attachment device 780 is also configured to receive a portion of the conduit 736 and anchor the conduit 736 to the leg 765 of the individual 766. As such, the leg attachment device 780 forms an anchor that helps secure the fluid collection assembly 700 to the vaginal region 768 of the individual 766.

In the illustrated embodiment, the leg attachment device 780 is configured to induce a bend in the conduit 736 which causes the conduit 736 to press the fluid collection assembly 700 into the vaginal region 768. For example, the conduit 736 may exhibit a natural shape, that is, a shape when no external forces are applied thereto. The conduit 736 may be bent such that the conduit 736 exhibits a bent shape that is different than the natural shape of the conduit 736. The bent shape may be selected such that the distance between the fluid collection assembly 700 and an anchor (e.g., the leg attachment device 780) is less when the conduit 736 exhibits the natural shape thereof. For instance, in the illustrated example, the natural shape of the conduit 736 may be straighter than the bent shape. The desire of the conduit 736 to return to the natural shape thereof may cause the conduit 736 to press the fluid collection assembly 700 into the vaginal region 768.

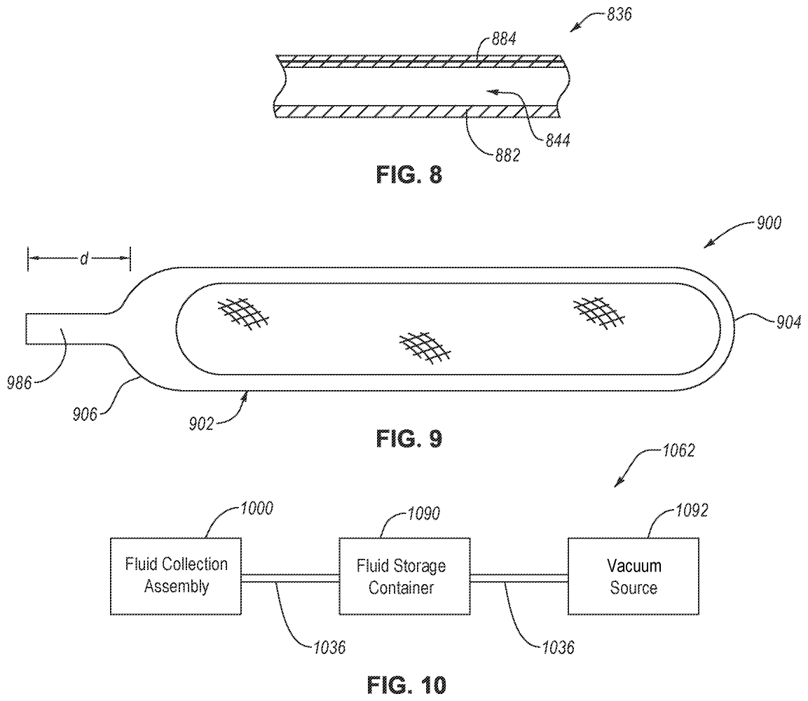

In some examples, the conduits disclosed herein may exhibit a natural shape that is coiled. When the conduit is disposed in the chamber of the fluid collection assembly, the coiled natural shape of the conduits may cause the fluid collection assemblies to exhibit an unsatisfactory shape that encourages leaks. When the conduit is disposed outside of the chamber, the coiled natural shape of the conduit may induce undesirable movement in the fluid collection assembly relative to the vaginal region which may cause leaks. As such, the conduits disclosed herein may include a shape memory material disposed in the conduit that may allow selectively changing the natural shape of the conduit. For example, FIG. 8 is a cross-sectional schematic of a conduit 836, according to an embodiment. The conduit 836 includes one or more side walls 882 defining a passageway 844. The conduit 836 may include a shape memory material 884 disposed in or attached to the side walls 882 of the conduit 836 or disposed in the passageway 844. The shape memory material 884 is configured to be shaped when an external force is applied thereto and maintain the shape thereof. The side walls 882 of the conduit 836 may be forced to exhibit a shape that generally corresponds to the shape of the shape memory material 884. Examples of shape memory materials may include a metal wire (e.g., a nitinol wire, a copper wire, a steel wire, or an aluminum wire) or other suitable materials. Further examples of shape memory materials are disclosed in PCT Patent Application No. PCT/US2020/042262 filed on Jul. 16, 2020, the disclosure of which is incorporated herein, in its entirety, by this reference.

The fluid collection assemblies disclosed herein may include leak prevention features other than or in addition to at least one of the wedged shaped porous material or the fluid outlet positioned on the back region of the fluid impermeable layer. For example, the leak prevention feature of the fluid collection assemblies may include an extension extending from the distal end region of the fluid impermeable layer that is configured to be positioned in the gluteal cleft. For example, FIG. 9 is a top plan view of a fluid collection assembly 900 that includes an extension 986, according to an embodiment. Except as otherwise disclosed herein, the fluid collection assembly 900 is the same or substantially similar to any of the fluid collection assemblies disclosed herein. For example, the fluid collection assembly 900 may include a fluid impermeable layer 902 including a proximal end region 904 and a distal end region 906 opposite the proximal end region 904.

The extension 986 is configured to be positioned in the gluteal cleft of an individual. For example, the extension 986 extends from the distal end region 906 of the fluid impermeable layer 902 (e.g., a portion of the fluid impermeable layer 902 that is closest to the gluteal cleft) which allows the extension 986 to be positioned in the gluteal cleft. Positioning the extension 986 in the gluteal cleft may facilitate alignment of the fluid collection assembly 900 relative to the vaginal region of the individual and may help secure the fluid collection assembly 900 to the individual, both of which prevent leaks. As such, the extension 986 is a leak prevention feature. The extension 986 may also limit stool moving from the anal region to the chamber by moving the distal end region 906 away from the gluteal cleft.

The extension 986 may exhibit any suitable shape that allows the extension 986 to be positioned in the gluteal cleft. In an example, as illustrated, the extension 986 exhibits a generally flat shape. The generally flat shape of the extension 986 allows the extension 986 to be positioned between the gluteal cleft while minimizing the distance that the extension 986 pushed the buttocks of the individual apart thereby increasing the individual's comfort. Also, the generally flat shape may increase the surface area of the extension 986 that contacts the buttocks of the individual which may better maintain the extension 986 in the gluteal cleft. In an example, the extension 986 may exhibit a generally cylindrical shape (e.g., generally rod-like shape) or a similar rounded shape (e.g., a shape exhibiting a generally oval or ellipsoidal cross-sectional shape). The generally cylindrical shape or rounded shape of the extension 986 may prevent the extension 986 from uncomfortably pressing into the buttock of the individual. The shape of the extension 986 may be selected based on the size and shape of the individual's anatomy and/or based on the individual's preference.

In an embodiment, the extension 986 may exhibit a curved (e.g., bent) shape. The curved shape of the extension 986 may allow the extension 986 to better conform to the shape of the anatomy of the individual than if the extension 986 was generally straight. For example, the gluteal cleft may extend along a generally curved path and the generally curved shape of the extension 986 may better correspond to the curved path of the gluteal cleft than if the extension 986 was generally straight. Configuring the extension 986 to generally correspond to the path of the gluteal cleft may prevent or at least inhibit the extension 986 extending out of the gluteal cleft which may increase the likelihood that the extension 986 becomes dislodged from the gluteal cleft.

The extension 986 may extend a distance d from the fluid impermeable layer 902. The distance d may be measured generally parallel to a longitudinal axis 122 of the fluid collection assembly 100 and/or parallel to a longitudinal axis of the extension 986. The distance d that the extension 986 extends from the fluid impermeable layer 902 may be about 1 cm or greater, about 2 cm or greater, about 3 cm or greater, about 5 cm or greater, about 7.5 cm or greater, about 10 cm or greater, about 12.5 cm or greater, about 15 cm or greater, about 17.5 cm or greater, about 20 cm or greater, about 22.5 cm or greater, about 25 cm or greater, or in ranges of about 1 cm to about 3 cm, about 2 cm to about 5 cm, about 3 cm to about 7.5 cm, about 5 cm to about 10 cm, about 7.5 cm to about 12.5 cm, about 10 cm to about 15 cm, about 12.5 cm to about 17.5 cm, about 15 cm to about 20 cm, about 17.5 cm to about 22.5 cm, or about 20 cm to about 25 cm. The distance d that the extension 986 extends from the fluid impermeable layer 902 may depend on a variety of factors. In an example, the distance d that the extension 986 extends from the fluid impermeable layer 902 may depend on the size of the anatomy the individual. For instance, the distance d that the extension 986 extends from the fluid impermeable layer 902 may be greater when used with a larger individual than with a smaller individual since the larger individual may have a larger (e.g., longer) gluteal cleft that can receive the extension 986 and the distance from the distal end region 106 to the gluteal cleft may be larger with the larger individual. In an example, the distance d that the extension 986 extends from the fluid impermeable layer 902 may depend on the individual's preference since some individuals may like extensions 986 that extend further from the fluid impermeable layer 902 than others. In either example, reversibly attaching the extension 986 to the fluid impermeable layer 902 allows the fluid impermeable layer 902 to be chosen depending on the size of the individual and/or the individual's preference.

Further examples of extensions are disclosed in U.S. Provisional Patent Application No. 63/241,562 filed on Sep. 8, 2021, the disclosure of which is incorporated herein, in its entirety, by this reference.

FIG. 10 is a block diagram of a fluid collection system 1062 for fluid collection, according to an embodiment. The fluid collection system 1062 includes a fluid collection assembly 1000, a fluid storage container 1090, and a vacuum source 1092. The fluid collection assembly 1000 may be the same or substantially similar to any of the fluid collection assemblies disclosed herein. The fluid collection assembly 1000, the fluid storage container 1090, and the vacuum source 1092 may be fluidly coupled to each other via one or more conduits 1036. For example, fluid collection assembly 1000 may be operably coupled to one or more of the fluid storage container 1090 or the vacuum source 1092 via the conduit 1036. The bodily fluids collected in the fluid collection assembly 1000 may be removed from the fluid collection assembly 1000 via the conduit 1036 which protrudes into the fluid collection assembly 1000. For example, an inlet of the conduit 1036 may extend into the fluid collection assembly 1000, such as to a reservoir therein. The outlet of the conduit 1036 may extend into the fluid collection assembly 1000 or the vacuum source 1092. Suction force may be introduced into the chamber of the fluid collection assembly 1000 via the inlet of the conduit 1036 responsive to suction (e.g., vacuum) force applied at the outlet of the conduit 1036.

The suction force may be applied to the outlet of the conduit 1036 by the vacuum source 1092 either directly or indirectly. The suction force may be applied indirectly via the fluid storage container 1090. For example, the outlet of the conduit 1036 may be disposed within the fluid storage container 1090 and an additional conduit 1036 may extend from the fluid storage container 1090 to the vacuum source 1092. Accordingly, the vacuum source 1092 may apply suction to the fluid collection assembly 1000 via the fluid storage container 1090. The suction force may be applied directly via the vacuum source 1092. For example, the outlet of the conduit 1036 may be disposed within the vacuum source 1092. An additional conduit 1036 may extend from the vacuum source 1092 to a point outside of the fluid collection assembly 1000, such as to the fluid storage container 1090. In such examples, the vacuum source 1092 may be disposed between the fluid collection assembly 1000 and the fluid storage container 1090.

The fluid storage container 1090 is sized and shaped to retain bodily fluids therein. The fluid storage container 1090 may include a bag (e.g., drainage bag), a bottle or cup (e.g., collection jar), or any other enclosed container for storing bodily fluids such as urine. In some examples, the conduit 1036 may extend from the fluid collection assembly 1000 and attach to the fluid storage container 1090 at a first point therein. An additional conduit 1036 may attach to the fluid storage container 1090 at a second point thereon and may extend and attach to the vacuum source 1092. Accordingly, a vacuum (e.g., suction) may be drawn through fluid collection assembly 1000 via the fluid storage container 1090. Bodily fluids, such as urine, may be drained from the fluid collection assembly 1000 using the vacuum source 1092.

The vacuum source 1092 may include one or more of a manual vacuum pump, an electric vacuum pump, a diaphragm pump, a centrifugal pump, a displacement pump, a magnetically driven pump, a peristaltic pump, or any pump configured to produce a vacuum. The vacuum source 1092 may provide a vacuum or suction to remove bodily fluids from the fluid collection assembly 1000. In some examples, the vacuum source 1092 may be powered by one or more of a power cord (e.g., connected to a power socket), one or more batteries, or even manual power (e.g., a hand operated vacuum pump). In some examples, the vacuum source 1092 may be sized and shaped to fit outside of, on, or within the fluid collection assembly 1000. For example, the vacuum source 1092 may include one or more miniaturized pumps or one or more micro pumps. The vacuum sources 1092 disclosed herein may include one or more of a switch, a button, a plug, a remote, or any other device suitable to activate the vacuum source 1092.

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or 2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly comprising:
a fluid impermeable layer including a proximal end region, a distal end region spaced from the proximal end region, a front region defining at least one opening, and a back region opposite the front region, the fluid impermeable layer defining at least a chamber and a fluid outlet;
at least one porous material disposed in the chamber and extending across the at least one opening;
one or more leak prevention features, the one or more leak prevention features including at least one of:
at least a portion of the at least one porous material extending across the at least one opening exhibiting a generally wedge-like shape; or
the fluid outlet positioned on the back region of the fluid impermeable layer, the fluid outlet positioned closer to the proximal end region of the fluid impermeable layer than to the distal end region.

2. The fluid collection assembly of claim 1, wherein the one or more leak prevention features includes the at least a portion of the at least one porous material extending across the at least one opening exhibiting a generally wedge-like shape.

3. The fluid collection assembly of claim 2, wherein the at least one porous material includes a fluid permeable inner layer, a fluid permeable outer layer, and at least one fluid permeable intermediate material disposed between a portion of the fluid permeable inner layer and the fluid permeable outer layer adjacent to the at least one opening.

4. The fluid collection assembly of claim 3, wherein the fluid permeable inner layer defines a drainage channel.

5. The fluid collection assembly of claim 4, wherein the at least one fluid permeable intermediate material exhibits a size and shape that corresponds to the drainage channel defined by the fluid permeable inner layer.

6. The fluid collection assembly of claim 1, wherein the one or more leak prevention features includes the fluid outlet positioned on the back region of the fluid impermeable layer.

7. (canceled)

8. The fluid collection assembly of claim 6, further comprising at least one conduit extending through the fluid outlet and into the chamber.

9. The fluid collection assembly of claim 8, wherein the at least one conduit includes:
a first conduit extending in the chamber in a direction that is generally parallel to a longitudinal axis of the fluid impermeable layer;
a second conduit extending through the fluid outlet and into the chamber in a direction that is generally perpendicular to the longitudinal axis; and
an L-shaped fitting that fluidly couples the first conduit to the second conduit.

10. The fluid collection assembly of claim 8, wherein the at least one conduit includes:
a first conduit extending in the chamber in a direction that is generally parallel to a longitudinal axis of the fluid impermeable layer;
a second conduit extending through the fluid outlet and into the chamber in a direction that is generally perpendicular to the longitudinal axis; and
an T-shaped fitting having a first opening, a second opening opposite the first opening, and a third opening between the first and second openings, wherein the first opening is connected to the first conduit, the second opening is closed, and the third opening is connected to the second conduit.

11. The fluid collection assembly of claim 8, wherein at least a portion of the at least one conduit includes a shape memory material disposed therein or attached thereto.

12. The fluid collection assembly of claim 1, wherein the one or more leak prevention features further includes an extension extending from the distal end region of the fluid impermeable layer.

13. A fluid collection system, comprising:
a fluid collection assembly including:
a fluid impermeable layer including a proximal end region, a distal end region spaced from the proximal end region, a front region defining at least one opening, and a back region opposite the front region, the fluid impermeable layer defining at least a chamber and a fluid outlet;
at least one porous material disposed in the chamber and extending across the at least one opening;
one or more leak prevention features, the one or more leak prevention features including at least one of:
at least a portion of the at least one porous material extending across the at least one opening exhibiting a generally wedge-like shape; or
the fluid outlet positioned on the back region of the fluid impermeable layer, the fluid outlet positioned closer to the proximal end region of the fluid impermeable layer than to the distal end region
a fluid storage container; and
a vacuum source;
wherein the chamber of the fluid collection assembly, the fluid storage container, and the vacuum source are in fluid communication with each other such that, when one or more bodily fluids are present in the chamber, a suction provided from the vacuum source to the chamber of the fluid collection assembly removes the one or more bodily fluids from the chamber and deposits the bodily fluids in the fluid storage container.

14. The fluid collection system of claim 13, further comprising:
a generally wedge shaped pillow configured to fit between legs, the generally wedge shaped pillow including an apex configured to be positioned proximate to a urethral opening and to press the portion of the fluid permeable outer layer extending across the at least one opening against the urethral opening.

15. The fluid collection system of claim 14, wherein:
the one or more leak prevention features includes the fluid outlet positioned on the back region of the fluid impermeable layer and the fluid collection assembly includes at least one conduit extending through the fluid outlet and into the chamber; and
the generally wedge shaped pillow defines a recess therethrough configured to have the at least one conduit positioned therethrough.

16. The fluid collection system of claim 14, wherein the generally wedged shaped pillow includes:
a compressible supporting member; and
a casing positioned or positionable over the compressible supporting member, the casing including a fluid impermeable layer.

17. The fluid collection system of claim 16, wherein the casing includes a fluid impermeable layer

18. The fluid collection system of claim 17, wherein the casing includes a fluid receiving layer disposed on an outer surface of the fluid impermeable layer.

19. (canceled)

20. The fluid collection system of claim 13, further comprising a leg attachment device configured to be attached to a leg and a portion of at least one conduit that is disposed outside of the chamber.

21. The fluid collection system of claim 13, wherein the fluid outlet is positioned on the back region of the fluid impermeable layer and the fluid collection assembly includes at least one conduit extending through the fluid outlet and into the chamber; and
further comprising a leg attachment device configured to be attached to a leg and a portion of at least one conduit that is disposed outside of the chamber.

22. The fluid collection system of claim 21, wherein the leg attachment device includes a strap.
